(19) United States
(12) Patent Application Publication
(10) Pub. No.: US 2010/0106043 A1
(43) Pub. Date: Apr. 29, 2010

(54) BRAIN FUNCTION PARAMETER MEASUREMENT SYSTEM AND METHOD

(75) Inventors: Peter Alexander Robinson, New South Wales (AU); Christopher John Rennie, New South Wales (AU)

Correspondence Address: BANNER & WITCOFF, LTD., TEN SOUTH WACKER DRIVE, SUITE 3000, CHICAGO, IL, 60606, US

(73) Assignee: BRC IP PTY LTD, Ultimo, New South Wales (AU)

(21) Appl. No.: 12/532,303

(22) PCT Filed: Apr. 4, 2008
(86) PCT No.: PCT/AU08/00490

§ 371 Date: Sep. 21, 2009

(30) Foreign Application Priority Data
Apr. 4, 2007 (AU) 2007901820

(51) Int. Cl. A61B 5/048

(52) U.S. Cl. 600/544

(57) ABSTRACT

A method of fitting a proposed model for electro encephalography spectra to data derived from EEG recordings, the method comprising the steps of: (a) inputting at least one spectral trace of electroencephalographic measurements; (b) inputting a series of parameters associated with the proposed model; (c) applying a non-linear fitting algorithm to the at least one spectral trace and the at least one series of parameters, wherein the non-linear fitting model preferably can include a series of constraints associated with predetermined ones of the series of parameters so as to constrain the parameters in a predetermined range.

### FIELD OF THE INVENTION

The present invention relates to the field of measurement of electroencephalograms (EEGs) and, in particular, the presenting invention discloses methods of determining parameters of brain function by fitting EEG spectra predicted by them to observed EEG spectra.

### BACKGROUND OF THE INVENTION

The measurement of brain dynamics often involves the measurement and analysis of electrical activity within the brain. Complex waveforms are known to be generated by the neuronal structures within the brain. The study of these complex waveforms has further helped in understanding the brain's operation and is routinely use in clinical practice, an as a research tool for probing psychological states and processes.

The provision of an accurate brain monitoring tool also allows for an effective individualised brain treatment device to provide subjects with an enhanced brain interaction tool.

### SUMMARY OF THE INVENTION

It is an object of the presenting invention to provide for an improved form of analysis of spectral data associated with the brain's electrical activity.

The present invention is directed to quantifying electrical activity within the brain in terms of physiological and anatomical parameters. Knowledge of these parameters, and the fact that no invasive surgery is required to obtain them, is of considerable utility for clinical practice and for brain science.

A specific application is to Personalized Medicine, which can make use of individual-subject parameters to improve diagnostic sensitivity and specificity, determination of disorder and subgroup, and treatment prediction and response. Another application is to the basing of Neurofeedback methods on these quantities to stimulate, modulate, and/or control brain activity and behavior. A further application is to Human-Computer Interactions and Robotics, where parameters measuring brain state can be used to facilitate the provision of information and assistance to the user by the computer or robot

The present invention provides a method of fitting a proposed EEG generation model to recorded electroencephalographic spectra, the method comprising the steps of: (a) inputting at least one spectral trace of electroencephalographic measurements; (b) inputting initial parameter values, as determined by prior investigation; and (c) applying a non-linear fitting method to the at least one spectral trace and the at least one series of parameters, wherein the non-linear fitting model preferably can include a series of constraints associated with predetermined ones of the series of parameters so as to constrain the parameters in a predetermined range, while adjusting them to optimise the fit between the resulting predictions of the model and the actual spectra observed.

The non-linear fitting algorithm preferably can include utilising a Levenberg-Marquardt type algorithm to fit the data to the algorithm. The non-linear fitting algorithm preferably can include a cost function which increases superlinearly once a constraint can be passed. The model preferably can include a total subcortical signal, a corticothalamic feedback, an electromyogram component, and a thalamic signal source. The thalamus signal preferably can include a specific or secondary relay component and a reticular component.

The initial parameter values are preferably determined by prior investigation of electroencephalographic spectra measurements.

The method can be used to monitor the effects of a medical dose to provide a measure of one of diagnostic sensitivity and specificity, determination of disorder and subgroup, or treatment prediction and response. The method can further be utilised to stimulate, modulate, and/or control brain activity and behaviour.

The derived parameters are preferably utilised to provide information or assistance to a user.

### BRIEF DESCRIPTION OF THE DRAWINGS

Preferred forms of the invention will now be described with reference to the accompanying drawings in which:

FIG. 1 illustrates the basic steps in the operation of the preferred embodiment;

FIG. 2 illustrates the formation of extra constraint information in accordance with the preferred embodiment; and

FIG. 3 illustrates a brain monitoring and feedback system utilising the steps of the preferred embodiment.

### DESCRIPTION OF PREFERRED AND OTHER EMBODIMENTS

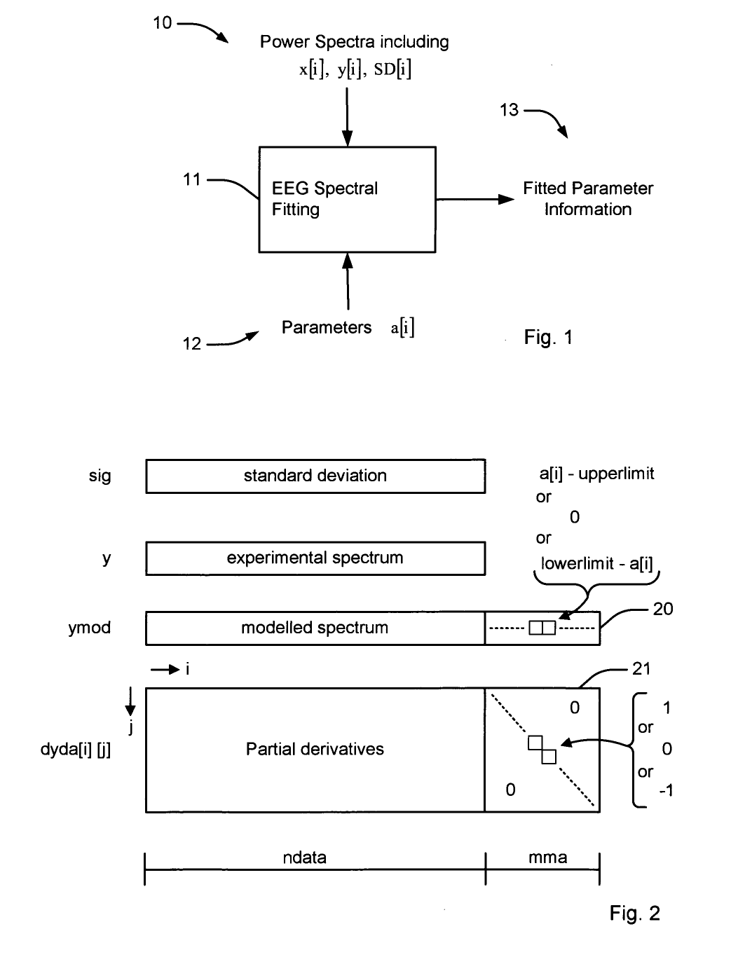

In the preferred embodiment, there is provided a system that uniquely fits the measured spectral data of an electroencephalograms or the like in accordance with a series of parameters. The overall structure of the program can be as illustrated in FIG. 1 wherein spectral data 10 is input to the program 11 in addition to a series of fitting parameters 12. The program outputs fitted parameter information 13.

Exemplary example uses of the system will be described hereinafter.

The preferred embodiment is based around an implementation of the routine mrqmin( . . . ) which implements a modified form of the Levenberg-Marquardt method for non-linear least squares curve fitting. The Levenberg-Marquardt method is fully explained in Chapter 15 of the well-known text “Numerical Recipes in C” by Press et al. (Cambridge University Press, Cambridge, 1992). The routine mrqmin ( . . . ) can have the following arguments.

| | |
|---|---|
| x[ ] | Array of frequency values; usually evenly spaced |
| y[ ] | Array of measured spectral powers corresponding to |
| | x[ ], in μV²/Hz |
| sig[ ] | Standard deviation (SD) of values in y[ ] |
| npt | Number of values in the arrays x[ ], y[ ], sig[ ] |
| a[ ] | Model parameters |
| ia[ ] | Flags whether parameters are fixed, or are free to |
| | be fitted |
| MA | Number of values in the arrays a[ ], ia[ ] |
| EvalModelFunc | Name of function that evaluates the theoretical |
| | spectrum corresponding to a[ ], plus its partial |
| | derivatives with respect to each of the parameters |
| | being fitted |
| alpha, covar | Auxiliary matrices, used by mrqmin ( . . . ) |
| χ² | A measure of the quality of the fit between measured |
| | and theoretical spectra |
| alamda | A value used to control initialisation and termination |
| | of fitting procedure; also a measure of step size |
| | during fitting |

The spectral input data are x[npt], y[nps], and sig[npt]. It is normally convenient to modify the SDs: first by a factor \sqrt{f} so that the high frequency tail will end up with smaller weights (this is like including a weighting factor 1/f in the equation for χ2); and second around specific frequencies like the mains frequency of 50 or 60 Hz where it might be desirable to enhance or diminish the weighting. Smoothing and log-transformation of y[ ] and sig[ ] can also be carried out depending on requirements.

The parameter values are a[MA] and ia[MA]. The values contained in the array a[ ], the model parameters, are described below. Some parameters must be constrained to a particular range during fitting, while others can be unconstrained, as described below. Some parameters (those that are being fitted) need to be given initial values, while others are fixed or are derived from fitted parameters.

The function EvalModelFunc( ) is described below.

During execution, the spectral arrays are initialised, suitable values are used to initialise a[ ] and ia[ ], the auxiliary matrices alpha[ ][ ] and covar[ ][ ] are initialised, and then iterative fitting can commence. Each iteration can involve outputting the current values of all relevant parameters, plotting or monitoring a superposition of the experimental and theoretical spectra, and calling the routine mrqmin( . . . ) to update the parameters.

As the fitting algorithm proceeds, χ2 decreases monotonically, and eventually approaches its global minimum, provided the initial parameter values were appropriate. When the values of χ2 appear to be approaching an asymptotic value, the iterations can be halted, and a full listing of all parameter values can be output for utilisation.

A number of important aspects of the method include:

### Model Parameters

The parameters, their nature and their initial values can be determined by experiment. Example parameter values are tabulated in below, showing alternative nomenclatures, and possible classification of each into fittable (optionally fitted or fixed), derived (calculated from other parameters), or fixed (constant).

| | Maths | | | Mini- | Maxi- | |
|---|---|---|---|---|---|---|
| Program | symbol | Class | Default | mum | mum | Units |
| gamma | γₑ | fittable | 130 | 40 | 400 | s⁻¹ |
| alpha | α | fittable | 75 | 10 | 200 | s⁻¹ |
| beta | β | derived | | 10 | 400 | s⁻¹ |
| bona | β/α | fixed | 4.0 | | | |
| S | S | fittable | 0.1 | 0 | 3 | |
| Gii | Gᵢᵢ | fittable | −7.0 | −35 | 1 | |
| Gese | GₑₛGₛₑ | fittable | 5.6 | 0 | 50 | |
| Gesre | GₑₛGₛᵣGᵣₑ | fittable | −2.8 | −30 | 2 | |
| Gsrs | GₛᵣGᵣₛ | fittable | −0.6 | −15 | 0.5 | |
| t0 | t₀ | fittable | 0.08 | 0.06 | 0.13 | s |
| an | n | fixed | 1 | | | |
| eta1 | η₁ | fixed | α | | | s⁻¹ |
| eta2 | η₂ | derived | | 10 | 400 | s⁻¹ |
| k0re | k₀rₑ | fixed | 3.0 | | | |
| lnorm | log₁₀(Pₙ) | fittable | 5.0 | | | log₁₀(rate² |
| | | | | | | Hz⁻¹) |
| Gee | Gₑₑ | derived | | 0 | 50 | |
| EMGa | A_{EMG} | fittable | 0.05 | 0 | 10 | rate² Hz⁻¹ |
| EMGf | f_{EMG} | fixed | 40.0 | | | Hz |
| EMGs | s | fixed | 2.0 | | | |
| X | X | derived | | 0 | 100 | |
| Y | Y | derived | | 18 | 60 | |
| Z | Z | derived | | 2 | 10 | |

The derived parameters have the following definitions:

$$β = {η}_{2} = ( β / α ) \times α$$

$${P}_{n} = π {\vert {φ}_{n} \vert}^{2} {G}_{es}^{2} {G}_{sn}^{2} / {r}_{e}^{2} , \\ {G}_{ee} = ( 1 - S ) ( 1 - {G}_{ii} ) - \frac{{G}_{es} {G}_{se} + {G}_{es} {G}_{sr} {G}_{re}}{1 - {G}_{sr} {G}_{rs}} , \\ X = \frac{{G}_{ee}}{1 - {G}_{ii}} , \\ Y = \frac{{G}_{es} {G}_{se} + {G}_{es} {G}_{sr} {G}_{re}}{( 1 - {G}_{sr} {G}_{rs} ) ( 1 - {G}_{ii} )} , \\ Z = - {G}_{sr} {G}_{rs} \frac{αβ}{{( α + β )}^{2}} .$$

### Constraints

Some parameters have constraints since it is not always easy for a single generic initialisation to achieve fits efficiently and reliably, especially when dealing with a variety of spectral shapes. The constraints express physiological limits. The non-linear fitting algorithm mrqmin ( . . . ), as described in “Numerical Recipes in C”, 2^{nd} edition, has no mechanism for imposing constraints, so the algorithm needs to be adapted, as described below.

The core idea is to extend the modelled spectrum or waveform by a number of values equal to the number of model parameters. This is done in EvalModelFunc ( ), which evaluates the model function and its partial derivatives as ymod [ ] and dyda [ ] [ ], respectively. These two arrays are used within the routine mrqcof ( . . . ) (see “Numerical Recipes in C”) to evaluate the covariance matrix and χ². In order to incorporate constraints, ymod [ ] and dyda [ ] [ ] are augmented with pseudo data 20, 21, as indicated in FIG. 2.

The extended data points 20, ymod [ndata+i], i=0, mma−1, are calculated thus:

if (a [i] is being fitted && constraints are active && a [i]>UpperLimit)

ymod[ndata+i]=a[i]−UpperLimit

else if (a [i] is being fitted && constraints are active && a [i]<LowerLimit)

ymod[ndata+i]=LowerLimit−a[i]

else

ymod[ndata+i]=0,

so ymod [ndata+i], as a function of a [i], is generally zero, but increases linearly whenever a constraint boundary is crossed.

The corresponding extension to dyda [ ] [ ]21 will mostly be zero, but elements on its diagonal can be non-zero. According to the definition of ymod [ ], non-zero values will be 1 when a [i] goes beyond an upper boundary, and −1 when a [i] goes beyond a lower boundary.

The augmented arrays ymod [ ] and dyda [ ] [ ] are combined in the routine mrqcof ( . . . ) with y [ ] and sig [ ] for the calculation of the auxiliary arrays and χ². The amended equations are as follows:

$$\begin{matrix}{χ}^{2} = {\sum }_{i = 0}^{ndata - 1} \frac{{( y [ i ] - y mod [ i ] )}^{2}}{{sig [ i ]}^{2}} + ndata {\sum }_{j = 0}^{mma - 1} \frac{{( 0 - y mod [ ndata + j ] )}^{2}}{{ScaleLength [ j ]}^{2}} & \\ {β}_{j} = {\sum }_{i = 0}^{ndata - 1} \frac{\begin{matrix}dyda [ i ] [ j ] \times \\ ( y [ i ] - y mod [ i ] )\end{matrix}}{{sig [ i ]}^{2}} + ndata \frac{\begin{matrix}dyda [ ndata + j ] [ j ] \times \\ ( 0 - y mod [ ndata + j ] )\end{matrix}}{{ScaleLength [ j ]}^{2}} & \\ {α}_{jk} = {\sum }_{i = 0}^{ndata - 1} \frac{\begin{matrix}dyda [ i ] [ j ] \times \\ dyda [ i ] [ k ]\end{matrix}}{{sig [ i ]}^{2}} + nda ta {δ}_{jk} \frac{{( dyda [ ndata + j ] [ j ] )}^{2}}{{ScaleLength [ j ]}^{2}} & \end{matrix}$$

Bearing in mind the definition of ymod [ ], it can be seen that the cost function χ² increases quadratically when a [i] crosses a constraint boundary, and that the constraints are applied in a way that is consistent with the ideas underlying the Levenberg-Marquardt technique.

Since some of the parameters might be fixed of derived from fitted parameters, the extension to αⱼₖ and βⱼ will be comprised of mfit×mfit, and mfit values, respectively, where mfit≦mma is the number of parameters being fitted.

The factor of ndata appears in the second term of the equations for χ², βⱼ and αⱼₖ. This factor is included so that contributions from the second term have roughly equal influence to that of the first term. The magnitude of this factor might be varied to alter the relative weights of the two terms.

The scheme allows for constraint of the mf it parameters being fitted. In addition to those parameters, there are typically others that are fixed, for which constraints are irrelevant; but there are also derived parameters like Gₑₑ and X which it may be desirable to constrain. There is no way to incorporate constraints on derived parameters within the scheme just described. Consequently, those parameters can be constrained by scaling χ² by a factor

$$1 + {\sum }_{i} \frac{{( {a}_{i} - Limit )}^{2}}{{ScaleLength}_{i}^{2}} ,$$

while not making any modifications to dyda [ ] [ ]. This achieves for derived parameters approximately the same end as the method above for constraining fitted parameters. By adding to the value of χ² it signals to the fitting algorithm when one of these parameters is outside its preferred range, but it says nothing about how best to minimize χ², since there are no corresponding terms in αⱼₖ or βⱼ. Nevertheless, the algorithm will respond to such modulations to χ².

### Brain Model Equations Evaluated by EvalModelFunc ( . . . )

The brain model utilised assumes (i) the cortex to be represented as a two-dimensional continuum, within which the excitatory synaptic activities (spikes per second) are represented by φₑ; (ii) that the total subcortical signal, φₛ, is the result of corticothalamic feedback of φₑ and a signal source φₙ at the thalamus; and (iii) that the thalamus consists of a specific or secondary relay component (subscript s) and a reticular component (subscript r). A further distinguishing characteristic of the model is that it fits an extra spectral component due to EMG.

The model utilised is similar to that described in P. A. Robinson, C. J. Rennie, J. J. Wright, H. Bahramali, E. Gordon, and D. L. Rowe. “Prediction of electroencephalographic spectra from neurophysiology.” Physical Review E, 63(2):021903, 2001 (Robinson et al.).

The foundation of the model is a set of equations, which encapsulate all aspects of neural electrophysiology that are salient to the scalp EEG. This is feasible since EEG recordings from the scalp show little spatial detail on scales less than a few centimetres, so the equations describing EEG need only involve local average characteristics of neural electrophysiology. In particular, it was shown in Robinson et al. that equations can be constructed for synaptic firing rates in terms of (i) average dendritic impulse functions L(t), which depend on synaptic and membrane time constants, and (ii) gain parameters G, which in turn depend on average synaptic strengths, the sensitivity of the area within neurons where action potentials are initiated, and the number of terminal synapses.

The spatial extent of excitatory (e) neurons can be much larger than that of inhibitory (i) neurons, and so the two populations are described by separate (but similar) equations and parameters. In particular there are several gains: Gₑₑ, Gₑᵢ, Gᵢᵢ, and Gᵢₑ (although Gₑₑ≈Gᵢₑ and Gₑᵢ≈Gᵢᵢ), as well as others arising when subcortical (s) pathways are considered.

It can be shown that the relationship between excitatory synaptic firing rates, φₑ, and the driving signal from the subcortex, φₛ, is

$$\frac{{φ}_{e} ( k , ω )}{{φ}_{s} ( k , ω )} = \frac{{G}_{es} L ( ω )}{{D}_{e} [ 1 - {G}_{ii} L ( ω ) ] - {G}_{ee} L ( ω )}$$

where

Dee=k²rₑ²+(1−iω/γₑ)².

This is the transfer function for the cortex, and is in terms of spatial (k) and temporal (ω) frequencies.

Furthermore, we assume that the subcortical signal is

φₛ=Pφₙ+Sφₑ

where

$$P = \frac{{L}_{s} {G}_{sn}}{1 - {L}_{s} {G}_{sr} {L}_{r} {G}_{re}} {e}^{iω {t}_{0} / 2}$$

$$S = \frac{{L}_{s} {G}_{se} + {L}_{s} {G}_{sr} {L}_{r} {G}_{rs}}{1 - {L}_{s} {G}_{sr} {L}_{r} {G}_{rs}} {e}^{iω {t}_{0}}$$

and all terms P, S, Lₛ, Lᵣ are functions of frequency ω. This form of φₛ can be combined with the cortical transfer function, with the result that the overall transfer function is,

$$\frac{{φ}_{e}}{{φ}_{n}} = \frac{{G}_{es} LP}{{D}_{e} ( 1 - {G}_{ii} L ) - {G}_{ee} L - {G}_{es} LS}$$

A further rearrangement is to expand Dₑ to make the spatial frequency of cortical activity, k, explicit:

$$\frac{{φ}_{e}}{{φ}_{n}} = \frac{{G}_{es} LP}{1 - {G}_{ii} L} \frac{1}{{k}^{2} {r}_{e}^{2} + {q}^{2} {r}_{e}^{2}}$$

The quantity of q²rₑ² is given by the following two equivalent expression,

$$\begin{matrix}{q}^{2} {r}_{e}^{2} = {( 1 - \frac{iω}{{γ}_{e}} )}^{2} - \frac{{G}_{ee} L + {G}_{es} LS}{1 - {G}_{ii} L} \\ = {( 1 - \frac{iω}{{γ}_{e}} )}^{2} - \frac{{G}_{ee} L}{1 - {G}_{ii} L} ( 1 + Ψ {L}_{s} {e}^{iω {t}_{0}} )\end{matrix}$$

$$Ψ = \frac{{G}_{es}}{{G}_{ee}} \frac{{G}_{se} + {G}_{sr} {L}_{r} {G}_{re}}{1 - {L}_{s} {G}_{sr} {L}_{r} {G}_{rs}}$$

Spatial smoothing is included to model the effects of volume conduction in the material overlying the brain (e.g. the skull, scalp, and cerebrospinal fluid). The spectral response to white noise (|φₙ(k, ω)|=const=|φₙ|²) with spatial smoothing (exp[−k²/k₀²]) is

$$\begin{matrix}P ( ω ) = \int \int {\vert {φ}_{e} / {φ}_{n} \vert}^{2} exp [ - {k}^{2} / {k}_{0}^{2} ] {d}^{2} k \\ = {\vert {φ}_{n} \vert}^{2} {\vert \frac{{G}_{es} LP}{1 - {G}_{ii} L} \vert}^{2} \int \int \frac{exp [ - {k}^{2} / {k}_{0}^{2} ]}{{\vert {k}^{2} {r}_{e}^{2} + {q}^{2} {r}_{e}^{2} \vert}^{2}} {d}^{2} k \\ = \frac{π {\vert {φ}_{n} \vert}^{2}}{{r}_{e}^{2}} {\vert \frac{{G}_{es} LP}{1 - {G}_{ii} L} \vert}^{2} \frac{Im [ exp ( {q}^{* 2} / {k}_{0}^{2} ) {E}_{1} ( {q}^{* 2} / {k}_{0}^{2} ) ]}{Im {q}^{2} {r}_{e}^{2}} .\end{matrix}$$

In the limit k₀→∞ this simplifies to

$$P ( ω ) = \frac{π {\vert {φ}_{n} \vert}^{2}}{{r}_{e}^{2}} {\vert \frac{{G}_{es} LP}{1 - {G}_{ii} L} \vert}^{2} \frac{Arg {q}^{2} {r}_{e}^{2}}{Im {q}^{2} {r}_{e}^{2}} ,$$

and the bipolar equivalent, for separation R and relative amplitudes C, is

$$P ( ω ) = \frac{π {\vert {φ}_{n} \vert}^{2}}{{r}_{e}^{2}} {\vert \frac{{G}_{es} LP}{1 - {G}_{ii} L} \vert}^{2} \frac{Arg {q}^{2} {r}_{e}^{2} + \frac{4 C}{1 + {C}^{2}} Im {K}_{0} ( qR )}{Im {q}^{2} {r}_{e}^{2}} .$$

The expressions above apply to the boundary-less case. For a spatially finite system activity can be described in terms of discrete modes, and as a result the spectrum is altered. In the case of a rectangular system with cyclical boundary conditions,

$$\begin{matrix}P ( ω ) = {\vert {φ}_{n} \vert}^{2} {\vert \frac{{G}_{es} LP}{1 - {G}_{ii} L} \vert}^{2} \frac{{( 2 π )}^{2}}{{L}_{x} {L}_{y}} {\sum }_{m , n = - M , - N}^{M , N} \frac{{e}^{- {k}_{m , n}^{2} / {k}_{0}^{2}}}{{\vert {k}_{m , n}^{2} {r}_{e}^{2} + {q}^{2} {r}_{e}^{2} \vert}^{2}} \\ = \frac{π {\vert {φ}_{n} \vert}^{2}}{{r}_{e}^{2}} {\vert \frac{{G}_{es} LP}{1 - {G}_{ii} L} \vert}^{2} \frac{4 π {r}_{e}^{2}}{{L}_{x} {L}_{y}} [ \begin{matrix}4 {\sum }_{m , n = 1 , 1}^{M , N} \frac{{e}^{- {k}_{m , n}^{2} / {k}_{0}^{2}}}{{\vert {k}_{m , n}^{2} {r}_{e}^{2} + {q}^{2} {r}_{e}^{2} \vert}^{2}} + \\ 2 {\sum }_{m = 1}^{M} \frac{{e}^{- {k}_{m , 0}^{2} / {k}_{0}^{2}}}{{\vert {k}_{m , 0}^{2} {r}_{e}^{2} + {q}^{2} {r}_{e}^{2} \vert}^{2}} + \\ 2 {\sum }_{n = 1}^{N} \frac{{e}^{- {k}_{0 , n}^{2} / {k}_{0}^{2}}}{{\vert {k}_{0 , n}^{2} {r}_{e}^{2} + {q}^{2} {r}_{e}^{2} \vert}^{2}} + \frac{1}{{\vert {q}^{2} {r}_{e}^{2} \vert}^{2}}\end{matrix} ] .\end{matrix}$$

For a rectangle of size Lₓ×L_{y} the discrete wavenumbers k_{m,n} are defined by

k_{m,n}²rₑ²=(2πmrₑ/Lₓ)²+(2πnrₑ/L_{y})².

More generally, the modes are chosen according to the geometery of the system. The EMG component is taken to be

$${P}_{EMG} ( ω ) = {A}_{EMG} \frac{{( ω / 2 π {f}_{EMG} )}^{2}}{{[ 1 + {( ω / 2 π {f}_{EMG} )}^{2} ]}^{s / 2 + 1}} ,$$

such that the EMG component has a maximum proportional to A_{EMG} at about f_{EMG}, and tends asymptotically to ω² at low frequencies and to ω^{-s} at high frequencies. The total spectral power is thus P(ω)+P_{EMG}(ω)

The routine EvalModelFunc ( . . . ) evaluates the total spectral power P(ω)+P_{EMG}(ω), together with its partial derivatives with respect to each of the parameters being fitted.

The reliability of the preferred embodiment can be improved by applying the above method multiple times for parameters scattered randomly around the initial values estimated from experiment, and then selecting consensus parameters from the collection of runs, after discarding any that are physiologically unrealistic. In the presence of experimental noise, this improvement reduces the likelihood that noise will lead to poor parameter estimates due to chance interactions with the specific initial values chosen. It also increases the likelihood of the method converging to a definite and physiologically realistic outcome, which may not occur for certain specific values of initial parameters.

Further, by modifying the equations of the fitted function appropriately, the method can be used to model additional components of the brain, including the brain stem, basal ganglia, and other structures.

Further, by modifying the equations of the fitted function appropriately, the method can be used to determine physiological, anatomical, neurochemical, and/or pharmacological parameters underlying other types of data on brain function and activity, including: evoked response potentials that result from short stimuli, steady state response potentials that result from sinusoidal stimuli, magnetoencephalographic measurements, functional magnetic resonance imaging signals, positron emission tomography data, and single photon emission computed tomography data.

The modelled parameter results can also be utilised in other different ways. These include: (1) Based on the response of a particular individual, determining which individual is best suited for treatment or a particular medicine on a personalised basis; (2) Utilising the fitted parameter information to provide an indicator of brain response to training activities and to thereby provide a feedback loop for brain/Body performance training in a personalised, targeted manner; (3) To provide response feedback for Brain or Brain/Body stimulation but various means including electrical, audio, visual, infrared and other forms of stimulation.

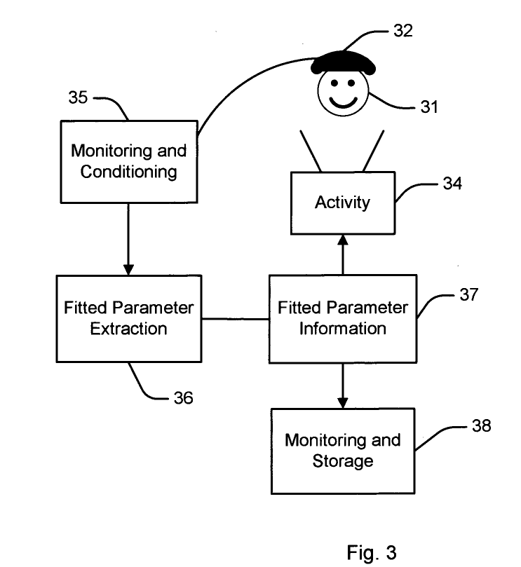

One form of utilisation system can be as illustrated schematically in FIG. 3, wherein a subject 31 undergoes various interactive tasks presented visually 34. The subject is monitored by EEG monitoring system 32. The monitored signals are input 35 where they are digitised, conditioned and translated into the spectral domain for forming the Power Spectra inputs to the EEG Spectral Fitting routines previously described with reference to FIG. 1. The output fitted parameter information 37 can be monitored and stored for analysis 38 as well as interactively feedback to the activity system 34 so as to provide enhanced feedback. In another form of system, the subject can be administered a medical dose and the method can be used to monitor the effects of a medical dose to provide a measure of one of diagnostic sensitivity and specificity, determination of disorder and subgroup, or treatment prediction and response. The method can further be utilised to stimulate, modulate, and/or control brain activity and behaviour. The derived parameters can also be utilised to provide information or assistance to a user.

The foregoing describes only preferred embodiments of the present invention. Modifications, obvious to those skilled in the art, can be made thereto without departing from the invention.

The methodologies described herein are, in one embodiment, performable by one or more processors that accept computer-readable (also called machine-readable) code containing a set of instructions that when executed by one or more of the processors carry out at least one of the methods described herein. Any processor capable of executing a set of instructions (sequential or otherwise) that specify actions to be taken are included. Thus, one example is a typical processing system that includes one or more processors. Each processor may include one or more of a CPU, a graphics processing unit, and a programmable DSP unit. The processing system further may include a memory subsystem including main RAM and/or a static RAM, and/or ROM. A bus subsystem may be included for communicating between the components. The processing system further may be a distributed processing system with processors coupled by a network. If the processing system requires a display, such a display may be included, e.g., a liquid crystal display (LCD) or a cathode ray tube (CRT) display. If manual data entry is required, the processing system also includes an input device such as one or more of an alphanumeric input unit such as a keyboard, a pointing control device such as a mouse, and so forth. The term memory unit as used herein, if clear from the context and unless explicitly stated otherwise, also encompasses a storage system such as a disk drive unit. The processing system in some configurations may include a sound output device, and a network interface device. The memory subsystem thus includes a computer-readable carrier medium that carries computer-readable code (e.g., software) including a set of instructions to cause performing, when executed by one or more processors, one of more of the methods described herein. Note that when the method includes several elements, e.g., several steps, no ordering of such elements is implied, unless specifically stated. The software may reside in the hard disk, or may also reside, completely or at least partially, within the RAM and/or within the processor during execution thereof by the computer system. Thus, the memory and the processor also constitute computer-readable carrier medium carrying computer-readable code. Furthermore, a computer-readable carrier medium may form, or be included in a computer program product. In alternative embodiments, the one or more processors operate as a standalone device or may be connected, e.g., networked to other processor(s), in a networked deployment, the one or more processors may operate in the capacity of a server or a client machine in server-client network environment, or as a peer machine in a peer-to-peer or distributed network environment. The one or more processors may form a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Note that while the diagrams only shows a single processor and a single memory that carries the computer-readable code, those in the art will understand that many of the components described above are included, but not explicitly shown or described in order not to obscure the inventive aspect. For example, while only a single machine is illustrated, the term “machine” shall also be taken to include any collection of machines that individually or jointly execute a set (or multiple sets) of instructions to perform any one or more of the methodologies discussed herein.

Thus, one embodiment of each of the methods described herein is in the form of a computer-readable carrier medium carrying a set of instructions, e.g., a computer program that are for execution on one or more processors, e.g., one or more processors that are part of whatever the device is, as appropriate. Thus, as will be appreciated by those skilled in the art, embodiments of the present invention may be embodied as a method, an apparatus such as a special purpose apparatus, an apparatus such as a data processing system, or a computer-readable carrier medium, e.g., a computer program product. The computer-readable carrier medium carries computer readable code including a set of instructions that when executed on one or more processors cause the processor or processors to implement a method. Accordingly, aspects of the present invention may take the form of a method, an entirely hardware embodiment, an entirely software embodiment or an embodiment combining software and hardware aspects. Furthermore, the present invention may take the form of carrier medium (e.g., a computer program product on a computer-readable storage medium) carrying computer-readable program code embodied in the medium. The software may further be transmitted or received over a network via a network interface device. While the carrier medium is shown in an exemplary embodiment to be a single medium, the term “carrier medium” should be taken to include a single medium or multiple media (e.g., a centralized or distributed database, and/or associated caches and servers) that store the one or more sets of instructions. The term “carrier medium” shall also be taken to include any medium that is capable of storing, encoding or carrying a set of instructions for execution by one or more of the processors and that cause the one or more processors to perform any one or more of the methodologies of the present invention. A carrier medium may take many forms, including but not limited to, non-volatile media, volatile media, and transmission media. Non-volatile media includes, for example, optical, magnetic disks, and magneto-optical disks. Volatile media includes dynamic memory, such as main memory. Transmission media includes coaxial cables, copper wire and fiber optics, including the wires that comprise a bus subsystem. Transmission media also may also take the form of acoustic or light waves, such as those generated during radio wave and infrared data communications. For example, the term “carrier medium” shall accordingly be taken to included, but not be limited to, solid-state memories, a computer product embodied in optical and magnetic media, a medium bearing a propagated signal detectable by at least one processor of one or more processors and representing a set of instructions that when executed implement a method, a carrier wave bearing a propagated signal detectable by at least one processor of the one or more processors and representing the set of instructions a propagated signal and representing the set of instructions, and a transmission medium in a network bearing a propagated signal detectable by at least one processor of the one or more processors and representing the set of instructions. It will be understood that the steps of methods discussed are performed in one embodiment by an appropriate processor (or processors) of a processing (i.e., computer) system executing instructions (computer-readable code) stored in storage. It will also be understood that the invention is not limited to any particular implementation or programming technique and that the invention may be implemented using any appropriate techniques for implementing the functionality described herein. The invention is not limited to any particular programming language or operating system.

1. A method of fitting a proposed model of electroencephalographic spectra to observed spectral data, the method comprising the steps of:
(a) inputting at least one spectral trace of electroencephalographic measurements;
(b) inputting a series of initial parameter values associated with the proposed model; and
(c) applying a non-linear fitting algorithm to said at least one spectral trace and said at least one series of parameters, wherein said non-linear fitting algorithm iteratively modifies parameter values to improve the quality of the fit, and includes a series of constraints associated with predetermined ones of said series of parameters so as to constrain the parameters in a predetermined range, and
(d) outputting the fitted parameters as a proposed model of the electroencephalographic spectra.

2. A method as claimed in claim 1 wherein said non-linear fitting algorithm includes utilising a Levenberg-Marquardt type algorithm to fit the data to the algorithm.

3. A method as claimed in claim 1 wherein the non-linear fitting algorithm includes a cost function which increases superlinearly once a constraint is passed.

4. A method as claimed in claim 1 wherein said model includes a total subcortical signal, a corticothalamic feedback, an electromyogram component and a thalamic signal source.

5. A method as claimed in claim 4 wherein the thalamus signal includes a specific or secondary relay component and a reticular component.

6. A method as claimed in claim 1 wherein said constraint increases linearly whenever a constraint boundary is crossed.

7. A method as claimed in claim 1 wherein said initial parameter values are determined by prior investigation of electroencephalographic spectra measurements.

8. A method as claimed in claim 1 wherein said method is used to monitor the effects of a medical dose to provide a measure of one of diagnostic sensitivity and specificity, determination of disorder and subgroup, or treatment prediction and response.

9. A method as claimed in claim 1 wherein the method is utilised to stimulate, modulate, and/or control brain activity and behaviour.

10. A method as claimed in claim 1 wherein the derived parameters are utilised to provide information or assistance to a user.

11. A method as claimed in claim 1 wherein said step (c) further includes the step of reducing the standard deviations of the observed spectral data in a predetermined frequency dependant manner.

12. A system for fitting a proposed model of electroencephalographic spectra to observed spectral data, the system comprising:
an electroencephalographic measurement unit measuring a subjects electroencephalographic response and outputting a spectral trace thereof; and
a parameter modelling unit connected to said spectral trace and applying a nonlinear fitting algorithm to determine a series of parameter model values to output a quality of fit of parameter values to the spectral trace for a predetermined brain model.

13. A system as claimed in claim 12 wherein said parameter modelling unit further includes a constraint unit which constrains predetermined ones of said series of parameter values to predetermined ranges

14. A method as claimed in claim 1 wherein said step (c) further includes the step of applying the non-linear fitting algorithm multiple times with different initial parameter values and selecting a set of consensus final parameters from the multiple application of the non-linear fitting algorithm.

15. A system as claimed in claim 12 wherein said model includes a total subcortical signal, a corticothalamic feedback, an electromyogram component and a thalamic signal source.

16. A system as claimed in claim 15 wherein the thalamus signal includes a specific or secondary relay component and a reticular component.

17. A system as claimed in claim 12 wherein said parameter modelling unit applies a frequency dependant attenuation of the standard deviations of the spectral trace.
